(19) United States
(12) Patent Application Publication
(10) Pub. No.: US 2004/0141935 A1
(43) Pub. Date: Jul. 22, 2004

(54) Reduction of hair growth

(76) Inventors: Peter Styczynski, Wrentham, MA (US); Rajeev Kumar Passi, Framingham, MA (US); Gurpreet S. Ahluwalia, Newton, MA (US); Douglas Shander, Acton, MA (US)

Correspondence Address: FISH & RICHARDSON PC, 225 FRANKLIN ST, BOSTON, MA, 02110, US

(21) Appl. No.: 10/347,987

(22) Filed: Jan. 21, 2003

(51) Int. Cl. A61K 7/06; A61K 7/15

(52) U.S. Cl. 424/70.1

(57) ABSTRACT

Hair growth can be reduced by topical application of a composition including an emulsion and a compound that inhibits hair growth. The emulsion (1) is prepared using a phase inversion procedure, (2) includes droplets having an average size of from 10 nm to 150 nm, (3) includes droplets sufficiently small that the composition is clear, (4) is in the form of a nanoemulsion, and/or (5) is an oil-in-water emulsion in which the compound that inhibits hair growth is dissolved in the water phase and the oil phase includes glyceryl isostearate.

### TECHNICAL FIELD

The invention relates to reducing hair growth in mammals, particularly for cosmetic purposes.

A main function of mammalian hair is to provide environmental protection. However, that function has largely been lost in humans, in whom hair is kept or removed from various parts of the body essentially for cosmetic reasons. For example, it is generally preferred to have hair on the scalp but not on the face.

Various procedures have been employed to remove unwanted hair, including shaving, electrolysis, depilatory creams or lotions, waxing, plucking, and therapeutic antiandrogens. These conventional procedures generally have drawbacks associated with them. Shaving, for instance, can cause nicks and cuts, and can leave a perception of an increase in the rate of hair regrowth. Shaving also can leave an undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair. Finally, antiandrogens—which have been used to treat female hirsutism—can have unwanted side effects.

It has previously been disclosed that the rate and character of hair growth can be altered by applying to the skin inhibitors of certain enzymes. These inhibitors include inhibitors of 5-alpha reductase, ornithine decarboxylase, S-adenosylmethionine decarboxylase, gamma-glutamyl transpeptidase, and transglutaminase. See, for example, Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; and Shander et al., U.S. Pat. No. 5,132,293.

α-Difluoromethylornithine (DFMO) is an inhibitor of omithine decarboxylase (ODC). A skin preparation containing DFMO (sold under the name Vaniqa®, has been approved by the Food and Drug Administration (FDA) for the treatment of unwanted facial hair growth in women. Its topical administration in a cream based vehicle has been shown to reduce the rate of facial hair growth in women. Vaniqa® facial cream includes a racemic mixture of the “D-” and “L-” enantiomers of DFMO (i.e., D,L-DFMO) in the monohydrochloride form at a concentration of 13.9% by weight active (15%, as monohydrochloride monohydrate). The recommended treatment regimen for Vaniqa® is twice daily. The cream base vehicle in Vaniqa® is set out in Example 1 of U.S. Pat. No. 5,648,394, which is incorporated herein by reference.

It generally takes about eight weeks of continuous treatment before the hair growth-inhibiting efficacy of Vaniqa® cream becomes apparent. Vaniqa® cream has been shown to decrease hair growth an average of 47%. In one study, clinical successes were observed in 35% of women treated with Vaniqa® cream. These women exhibited marked improvement or complete clearance of their condition as judged by physicians scoring a decrease in visibility of facial hair and a decrease in skin darkening caused by hair. Another 35% of the women tested experienced some improvement in their condition. However, there were some women who exhibited little or no response to treatment.

Accordingly, although Vaniqa® cream is an effective product, it would be even more effective if it provided an earlier onset of hair growth inhibition (i.e., exhibited efficacy earlier than eight weeks) and/or exhibited an increased clinical success rate (i.e., exhibited efficacy in a greater percentage of users).

The stratum corneum serves as a barrier to the influx of pathogens and toxins and the efflux of physiological fluids. The envelopes of the cells in the stratum corneum consists mainly of polar lipids, such as ceramides, sterols and fatty acids while the cytoplasm of the stratum corneum cells remains polar and aqueous. Poor transdermal penetration of some drugs has, until now, frustrated attempts to deliver clinically significant doses by the topical route.

Molecules that are identical to each other in chemical structural formula and yet are not superimposable upon each other are enantiomers. In terms of their physiochemical properties enantiomers differ only in their ability to rotate the plane of plane-polarized light, and this property is frequently used in their designation. Those enantiomers that rotate plane-polarized light to the right are termed dextrorotatory, indicated by either a (+)- or d- or D- before the name of the compound; those that rotate light to the left are termed laevorotatory indicated by a (−)- or l- or L- prefix. A racemic mixture is indicated by either a (±)- or d,l- or D,L- prefix. By another convention (or nomenclature), the R,S or the sequence rule can be used to differentiate enantiomers based on their absolute configuration. Using this system the L-DFMO corresponds to the R-DFMO, and the D-DFMO corresponds to the S-DFMO. Enantiomers are physiochemically similar in that they have similar melting points, boiling points, relative solubility, and chemical reactivity in an achiral environment. A racemate is a composite of equal molar quantities of two enantiomeric species, often referred to as the DL-form. Individual enantiomers of chiral molecules may possess different pharmacological profiles, i.e., differences in pharmacokinetics, toxicity, efficacy, etc.

### SUMMARY

The present invention provides a method (typically a cosmetic method) of reducing hair growth. The method includes applying to the skin, in an amount effective to reduce hair growth, a dermatologically acceptable composition comprising an emulsion including a compound that inhibits hair growth.

In one aspect of the invention, the emulsion has been prepared using a phase inversion procedure. By “phase inversion procedure”, we mean an emulsion that undergoes a phase inversion from either an oil-in-water emulsion to a water-in-oil emulsion or from a water-in-oil emulsion to an oil-in-water emulsion at a certain temperature, called the Phase Inversion Temperature.

In another aspect of the invention, one phase (for example, the water phase) of the emulsion includes droplets of the other phase (for example, the oil phase) having an average size of from 10 nm to 150 nm, and preferably, from 25 nm to 100 nm. Droplet size distribution can be measured by using Photone Correlation Spectroscopy as described by Diec et. al., (C&T, Vol. 116, pp. 61-66, 2001).

In another aspect of the invention, one phase of the emulsion includes droplets of the other phase sufficiently small that the composition is clear. By “clear”, we mean transparent to the naked eye.

In another aspect of the invention, the emulsion is a nanoemulsion.

In another aspect of the invention, the water phase of the emulsion includes the compound that inhibits hair growth and the oil phase of the emulsion includes glyceryl isostearate. In some embodiments the oil phase further includes an emulsifier and an emollient.

A preferred compound that inhibits hair growth is α-Difluoromethylornithine (DFMO). Preferably the DFMO comprises at least about 80%, more preferably at least about 90%, and most preferably at least 95%, L-DFMO. Ideally, the DFMO is substantially optically pure L-DFMO. “Substantially optically pure” means that the DFMO comprises at least 98% L-DFMO. “Optically pure” L-DFMO means that the DFMO comprises essentially 100% L-DFMO.

The preferred composition includes about 0.1% to about 30%, preferably about 1% to about 20%, and more preferably about 5% to about 15%, by weight of the compound that inhibits hair growth.

In some preferred embodiments, the emulsion is an oil-in-water emulsion and the composition includes from 0.59% to 50%, more preferably from 1% to 20%, of the oil phase by weight and from 40% to 99%, more preferably from 50% to 80%, of the water phase by weight.

The present investigation also provides topical compositions including a dermatologically or cosmetically acceptable vehicle and a compound that inhibits hair growth. The composition includes an emulsion (1) prepared using a phase inversion temperature procedure, (2) including droplets having an average size of from 10 nm to 150 nm, (3) including droplets sufficiently small that the emulsion is clear, (4) in the form of a nanoemulsion, and/or (5) is an oil-in-water emulsion in which the compound that inhibits hair growth is dissolved in the water phase and the oil phase includes glyceryl isostearate.

The present invention also provides a method of making a topical composition used for reducing hair growth using a phase inversion procedure.

The compositions preferably have an enhanced efficacy relative to similar compositions not including the emulsions discussed above. This enhanced efficacy can manifest itself, for example, in earlier onset of hair growth inhibiting activity, greater reduction of hair growth rate, and/or greater number of subjects demonstrating reduced hair growth.

Other features and advantages of the invention will be apparent from the description and from the claims.

### DETAILED DESCRIPTION

A preferred composition includes a compound that inhibits hair growth and a vehicle in the form of a nanoemulsion prepared using a phase inversion procedure. The composition may be a solid, liquid, or cream. The composition may be, for example, a cosmetic and dermatologic product in the form of an, for example, ointment, lotion, foam, cream, gel, or solution. The composition may also be in the form of a shaving preparation or an aftershave. The vehicle itself can be inert or it can possess cosmetic, physiological and/or pharmaceutical benefits of its own.

A preferred compound that inhibits hair growth is DFMO, which may be optically pure L-DFMO. Optically pure L-DFMO can be prepared by known methods. See, for example, U.S. Pat. No. 4,309,442; Gao et al., Ann. Pharm. Fr. 52(4):184-203 (1994); Gao et al., Ann. Pharm. Fr. 52(5):248-59 (1994); and Jacques et al., Tetrahedron Letters, 48:4617 (1971), all of which are incorporated by reference herein.

Other examples of compounds that inhibit hair growth include inhibitors of 5-α-reductase, antiandrogen compounds, and androgen receptor agents (see U.S. Pat. No. 4,885,289); other inhibitors of omithine decarboxylase, (see U.S. Pat. No. 4,720,489); inhibitors of S-adenosyl methionine decarboxylase (see U.S. Pat. No. 5,132,293); inhibitors of γ-glutamyl transpeptidase (see U.S. Pat. No. 5,096,911); inhibitors of adenylosuccinate synthetase (see U.S. Pat. No. 5,095,007); inhibitors of aspartate transcarbamylase (see U.S. Pat. No. 5,095,007); inhibitors of transglutaminase (see U.S. Pat. No. 5,143,925); inhibitors of L-asparagine synthetase (see U.S. Pat. No. 5,444,090); pantothenic acid and its analogues (see U.S. Pat. No. 5,364,885); sulfhydryl reactive compounds (see U.S. Pat. No. 5,411,991); inhibitors of lipoxygenase (see U.S. Pat. No. 6,239,170); inhibitors of cyclooxygenase (see U.S. Pat. No. 6,248,751); inhibitors of nitric oxide synthetase (U.S. Pat. No. 5,468,476); inhibitors of ornithine amino transferase (see U.S. Pat. No. 5,474,763); inhibitors of cysteine synthesis pathway enzymes (see U.S. Pat. No. 5,455,234); inhibitors of protein kinase C (see U.S. Pat. No. 5,554,608); catechin compounds (see U.S. Pat. No. 5,674,477); green tea polyphenols (see U.S. Pat. No. 5,776,442); non-steroidal angiogenesis suppressors (see U.S. Pat. No. 6,093,748); inhibitors of arginase (see U.S. Pat. No. 5,728,736); inhibitors of the metabolic pathway for the conversion of glucose to acetyl-CoA (see U.S. Pat. No. 5,652,273); compounds that inhibit the formation of glycoprotein, proteoglycans, and glycosaminoglycans (see U.S. Pat. No. 5,908,867); inhibitors of matrix metalloproteinase (see U.S. Pat. No. 5,962,466); inhibitors of the cholesterol synthesis pathway (see U.S. Pat. No. 5,840,752); inhibitors of DNA topoisomerase (see U.S. Pat. No. 6,037,326); inhibitors of aminoacyl-tRNA synthetase (see U.S. Pat. No. 5,939,458); inhibitors of the hypusine biosynthesis pathway (see U.S. Pat. No. 6,060,471); compounds that activate androgen conjugation (see U.S. Pat. No. 5,958,946); inhibitors of alkaline phosphatase (see U.S. Pat. No. 6,020,006); inhibitors of protein tyrosine kinase (see U.S. Pat. No. 6,121,269); and compounds that increase cellular ceramide levels (see U.S. Pat. No. 6,235,737). Examples of the above compounds can be found in the corresponding patents listed above. Specific examples include cyproterone acetate; progesterone; acivicin; anthglutin; L-alanosine; guanidino-succinic acid; ethacrynic acid; D-pantothenic acid; pantoyl alcohol; gabaculin; canaline; isonicotinic acid; verapamil; phentolamine; pentosan polysulfate; nafoxidine; tripelennamine; octapine; phloretin; argaric acid; simvastatin; atorvastatin; lovastatin; fluvastatin; mevastatin; N^{G}-methyl-L-arginine; N^{G}-nitro-L-arginine; benzoyl-L-argininamide; L-argininamide; quercetin; apigenin; nordihydroguaratic acid (NDGA); ketoprofen; naproxen; tolmetin; diclofenac; diflunisal; sulindac; thiosalicylic acid; cysteamine; diethyldithiocarbamic acid; D-penicillamine; N-acetyl-L-cysteine; bathocuproine; enalapril; tamoxifen; cimetidine; mycophenolic acid; tetracycline; doxycycline; minocycline; thioridazine; trifluoperizine; 1-(5-isoquinolinylsulfonyl)-2-methylpiperazine; glycyrrhetinic acid; epigallocatechin gallate; epicatechin gallate; epigallocatechin; epicatechin; fusidic acid; and nitroso-acetyl-penicillamine. The patents listed above, including the specific examples of compounds mentioned in the patents, are incorporated by reference.

The compositions may include more than one compound that inhibits hair growth.

The nanoemulsion may be an oil-in-water emulsion or a water-in-oil emulsion.

The water phase includes water and may optionally include hydrophilic solvents such as ethyl alcohol, isopropanol, acetone, diethylene glycol, ethylene glycol, glycerol, dimethyl sulfoxide, and dimethyl formamide. The water phase generally also includes the compound that inhibits hair growth, provided that the active compound is hydrophilic and soluble therein. The water phase may also include other water-soluble components such as detergents or emulsifiers, urea, film forming agents, hyaluronic acid, or other agents that could provide aesthetics or efficacy benefits in synergistic combination with one or more of hair growth inhibitors. The water phase may constitute, for example, from 40% to 99% of the composition by weight.

The oil phase may include, for example, (1) esters of an alkanecarboxylic acid having from 3 to 30 carbon atoms and alcohols having from 3 to 30 carbon atoms, and (2) esters of aromatic carboxylic acids and alcohols having from 3 to 30 carbon atoms. Specific examples include glyceryl isostearate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-hexyldecyl stearate, 2-octyldodecyl palmitate, oleyl oleate, oleyl erucate, erucyl oleate. and erucyl erucate. The oil phase may also include components such as 2-ethylhexyl isostearate, octyldodecanol, isotridecyl isononanoate, isoeicosane, 2-ethylexyl cocate, is C12-15 alkyl benzoate, caprylic-capric acid triglyceride, and dicaprylyl ether. The oil phase may constitute, for example, from 1% to 30% of the composition by weight.

The composition may also include one or more emollients which, depending on their solubility, may be part of the water phase or the oil phase. Examples of emollients that may be used include stearyl alcohol, mink oil, cetyl alcohol, oleyl alcohol, isopropyl laurate, polyethylene glycol, olive oil, petroleum jelly, palmitic acid, oleic acid, cyclomethicone, and myristyl myristate. The composition may include, for example, from 0.5% to 20% of emollients by weight.

The composition may also include an emulsifier. The emulsifier may, for example, have the formula R-O—[(CH₂)ₓ—O]ₙ—H, wherein x is 2 or 3, n is from 5 to 50, and R is an alkyl or alkylene group having from 5 to 30 carbon atoms. Examples include polyethylene glycol (13-20) stearyl ether, polyethylene glycol (12-20) isostearyl ether, polyethylene glycol (13-20) cetyl ether, polyethylene glycol (12-15) oleyl ether, polyethylene glycol (12) lauryl ether, polyethylene glycol (13-20) cetylstearyl ether, polyethylene glycol (20-25) stearate, polyethylene glycol (12-25) isostearate, polyethylene glycol (12-20) oleate, and polyethylene glycol (20-23) glyceryl laurate.

The composition may also include compounds that enhance the penetration of the compound that reduces hair growth into the skin. Examples of such compounds for use, in particular, with DFMO are described in U.S. Ser. No. 10/198,456, U.S. Ser. No. 10/198,536, and U.S. Provisional Serial No. 60/372,555. All of these applications are owned by the same owner as the present application and are hereby incorporated by reference. Skin penetration enhancers described in the applications include polyoxyethylene ethers having the chemical formula (R(OCH₂CH₂)_{b}OH, where R is a saturated or unsaturated alkyl group including from 6 to 22 carbon atoms and b is from 2 to 200; mineral oil; cis-fatty acids; fatty acid esters; terpenes; non-ionic surfactants; 2-n-nonyl-1,3-dioxolane; film-forming agents; dipropylene glycol dimethylether; cetiol; capric/caprylic triglyceride; fatty alcohols, triacetin monocaprylate/caprate; and 1-dodecyl-2-pyrrolidone.

The composition may include, for example, from 0.1% to 15% of one or more skin penetration enhancers by weight.

To prepare an emulsion using phase inversion, an oil phase and a water phase are selected that undergo a phase inversion (for example, from an water-in-oil emulsion to a oil-in-water emulsion) as the temperature of the emulsion drops from an elevated temperature to room temperature. The temperature at which this occurs is the phase inversion temperature. See, for example, the procedures described in Foster et.al., Phase Inversion Emulsification (C&T 106, pp. 49-52, 1991) and Diec et.al., PIT Microemulsions with Low Surfactant Content (C&T 116, pp. 61-66, 2001). In a preferred procedure, the oil phase and water phase are heated separately to or above the phase inversion temperature, combined, and allowed to cool to undergo the phase inversion.

The following general procedures can be used to prepare the subsequent specific examples.

For liquid compositions, the water phase included DI water, glycerin, and DFMO, and the oil phase included isoceteth-20, glyceryl isostearate, and an emollient oil such as dicaprylyl ether. The water phase and the oil phase were heated separately to 85-90° C. At 85-90° C., the water phase was added into the oil phase and mixed for 20 minutes to form a water-in-oil emulsion. The emulsion inverted to a clear oil-in-water emulsion on cooling down to room temperature at a rate of about 1 degree/minute. The preservatives were added, after cooling to room temperature.

For lotions, the phase inversion nanoemulsion was prepared in the same general way. However, in the lotion the emulsion was thickened with Acid Stable Base powder^{-R}, which is a combination of hydroxypropyl starch phosphate (and) acrylates/vinyl isodecanoate crosspolymer (and) xanthan gum (and) ceratonia siliqua gum (and) cyamopsis tetragonoloba (guar) gum. The aesthetics were adjusted with AM (Aesthetic Modifier) 200 [water (and) cyclomethicone (and) PEG-8 (and) phospholipids (and) polyphosphorylcholine glycol acrylate], AM300 [water (and) phenyl trimethicone (and) cylcomethicone (and) phospholipids (and) dimethiconol (and) polyphosphorylcholine glycol acrylate], and AM400 [water (and) hydrogenated polyisobutene (and) PEG-8 (and) cylcomethicone (and) phospholipids (and) polyphosphorylcholine glycol acrylate].

The acid stable powder was added to the emulsion and mixed with a high-speed stirrer until completely hydrolyzed (a homogenizer may be used, if needed). The pH was adjusted to 4.5-5.0 with triethanolamine and mix again at high speed. The AM200, AM300, and AM400 were added and mixed until the system is smooth and homogeneous.

An emulsion also was prepared the same way as described above for the liquid above. But the emulsion was then packed with propellant A-46 (19.1% propane +80.9% isobutane) in aluminum can, and was dispensed as quick-breaking foam.

### EXAMPLE 1

| INCI Name | w/w (%) |
|---|---|
| DFMO | 1.00 |
| Glycerol (Glycerin) | 3.00 |
| Isoceteth-20 | 4.60 |
| Glyceryl Isostearate | 2.40 |
| Dicaprylyl ether | 5.00 |
| Preservative, fragrance and color | q.s. |
| Water | to 100.00 |

### EXAMPLE 2

| INCI Name | w/w (%) |
|---|---|
| DFMO | 1.00 |
| Glycerol (Glycerin) | 3.00 |
| Isoceteth-20 | 4.60 |
| Glyceryl Isostearate | 2.40 |
| Caprylic/Capric Triglyceride | 5.00 |
| Preservative, fragrance and color | q.s. |
| Water | to 100.00 |

### EXAMPLE 3

| INCI Name | w/w (%) |
|---|---|
| DFMO | 1.00 |
| Glycerol (Glycerin) | 3.00 |
| Isoceteth-20 | 4.60 |
| Glyceryl Isostearate | 2.40 |
| Coco-Caprylate/Caprate | 5.00 |
| Preservative, fragrance and color | q.s. |
| Water | to 100.00 |

### EXAMPLE 4

| INCI Name | w/w (%) |
|---|---|
| DFMO | 1.00 |
| Glycerol (Glycerin) | 3.00 |
| Isoceteth-20 | 4.60 |
| Glyceryl Isostearate | 2.40 |
| Dicaprylyl ether | 15.00 |
| Preservative, fragrance and color | q.s. |
| Water | to 100.00 |

### EXAMPLE 5

| INCI Name | w/w (%) |
|---|---|
| DFMO | 1.00 |
| Glycerol (Glycerin) | 3.00 |
| Isoceteth-20 | 4.60 |
| Glyceryl Isostearate | 2.40 |
| Oleyl Alcohol | 5.00 |
| Preservative, fragrance and color | q.s. |
| Water | to 100.00 |

### EXAMPLE 6

| INCI Name | w/w (%) |
|---|---|
| DFMO | 1.00 |
| Glycerol (Glycerin) | 3.00 |
| Isoceteth-20 | 4.60 |
| Glyceryl Isostearate | 2.40 |
| Bis(2-ethylhexyl) carbonate | 5.00 |
| Preservative, fragrance and color | q.s. |
| Water | to 100.00 |

### EXAMPLE 7

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Preservative, fragrance and color | q.s. |
| Water | to 100.00 |

### EXAMPLE 8

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Caprylic/Capric Triglyceride | 3-15 |
| Preservative, fragrance and color | q.s. |
| Water | to 100.00 |

### EXAMPLE 9

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Coco-Caprylate/Caprate | 3-15 |
| Preservative, fragrance and color | q.s. |
| Water | to 100.00 |

### EXAMPLE 10

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Preservative, fragrance and color | q.s. |
| Water | to 100.00 |

### EXAMPLE 11

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Oleyl Alcohol | 3-15 |
| Preservative, fragrance and color | q.s. |
| Water | to 100.00 |

### EXAMPLE 12

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Bis(2-ethylhexyl) carbonate | 3-15 |
| Preservative, fragrance and color | q.s. |
| Water | to 100.00 |

### EXAMPLE 13

| INCI Name | w/w (%) |
|---|---|
| DFMO | 0.5-15.00 |
| Glycerol (Glycerin) | 0-5 |
| Sodium Chloride | 0.5-5.0 |
| Isoceteth-20 | 3-7 |
| Glyceryl Isostearate | 1.5-5 |
| Dicaprylyl ether | 3-15 |
| Cyclomethicone | 0.5-5 |
| Preservative, fragrance and color | q.s. |
| Water | to 100.00 |

### EXAMPLE 14

| INCI Name | w/w (%) |
|---|---|
| DFMO | 0.5-15.00 |
| Glycerol (Glycerin) | 0-5 |
| PEG 25 Stearate | 3-7 |
| Glyceryl Isostearate | 1.5-5 |
| Dicaprylyl ether | 3-15 |
| Preservative, fragrance and color | q.s. |
| Water | to 100.00 |

### EXAMPLE 15

| INCI Name | w/w (%) |
|---|---|
| DFMO | 0.5-15.00 |
| Glycerol (Glycerin) | 0-5 |
| PEG 20 Sorbitan Isostearate | 3-7 |
| Sorbitan Isostearate | 1.5-5 |
| Dicaprylyl ether | 3-15 |
| Cyclomethicone | 1-5 |
| Preservative, fragrance and color | q.s. |
| Water | to 100.00 |

### EXAMPLE 16

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Oleyl Alcohol | 1-5 |
| Preservative, fragrance and color | q.s. |
| Water | to 100.00 |

### EXAMPLE 17

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Diethylcyclohexane | 3-15 |
| Preservative, fragrance and color | q.s. |
| Water | to 100.00 |

### EXAMPLE 18

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| cis-Fatty acids* | 0.5-10% |
| Preservative, fragrance and color | q.s. |
| Water | to 100.00 |

*cis-fatty acids may include but are not restricted to oleic acid, palmitoleic acid, petroselenic acid and erucic acid.

### EXAMPLE 19

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Terpenes* | 0.5-10% |
| Preservative, fragrance and color | q.s. |
| Water | to 100.00 |

*A terpene may include but is not restricted to the following compounds: nerolidol, menthone, menthol, 1,8-cineole, terpineol, D-limonene, linalool and carvacrol.

### EXAMPLE 20

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| *Polyoxyethylene ethers | 0.5-10% |
| Preservative, fragrance and color | q.s. |
| Water | to 100.00 |

*A polyoxyethylene ether may include but is not restricted to Brij-30, Brij-72, Brij-78, Brij-92 and Brij-700.

### EXAMPLE 21

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Polyoxyethylene sorbitans* | 0.5-10% |
| Preservative, fragrance and color | q.s. |
| Water | to 100.00 |

*A polyoxyethylene sorbitan may include but is not restricted to Tween-20, Tween-40, Tween-60 and Tween-80.

### EXAMPLE 22

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| SEPA* | 0.5-10% |
| Preservative, fragrance and color | q.s. |
| Water | to 100.00 |

*SEPA is also known as 2-n-nonyl-1,3-dioxolane.

### EXAMPLE 23

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Film-forming agents* | 0.5-10% |
| Preservative, fragrance and color | q.s. |
| Water | to 100.00 |

*A film forming agent may include but is not restricted to Methocel and Dermacry-LT (Dow, Midland, MI).

### EXAMPLE 24

| INCI Name | w/w (%) |
|---|---|
| Water | q.s. to 100 |
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Dipropylene glycol | 0.5-10% |
| dimethylether | |
| Preservative, fragrance and color | q.s. |
| Water | to 100.00 |

### EXAMPLE 25

| INCI Name | w/w (%) |
|---|---|
| Water | q.s. to 100 |
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Lauryl alcohol | 0.5-10% |
| Preservative, fragrance and color | q.s. |
| Water | to 100.00 |

### EXAMPLE 26

| INCI Name | w/w (%) |
|---|---|
| Water | q.s. to 100 |
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Glyceryl triacetate (triacetin) | 0.5-10% |
| Preservative, fragrance and color | q.s. |
| Water | to 100.00 |

### EXAMPLE 27

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| 1-dodecyl-2-pyrrolidanone | 0.5-10% |
| Preservative, fragrance and color | q.s. |
| Water | to 100.00 |

### EXAMPLE 28

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Monocaprylate/Caprate | 0.5-10% |
| Preservative, fragrance and color | q.s. |
| Water | to 100.00 |

### EXAMPLE 29

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Isopropyl myristate | 0.5-10% |
| Preservative, fragrance and color | q.s. |
| Water | to 100.00 |

### EXAMPLE 30

| INCI Name | w/w (%) |
|---|---|
| DFMO | 0.5-15.00 |
| Glycerol (Glycerin) | 0-5 |
| Isoceteth-20 | 3-7 |
| Glyceryl Isostearate | 1.5-5 |
| Dicaprylyl ether | 3-15 |
| Hydroxypropyl starch phosphate (and) | 1.0-5.0 |
| acrylates/vinyl isodecanoate crosspolymer | |
| (and) xanthan gum (and) ceratonia siliqua | |
| gum (and) cyamopsis tetragonoloba (guar) | |
| gum.¹ | |
| water (and) cyclomethicone (and) PEG-8 | 1-10 |
| (and) phospholipids (and) | |
| polyphosphorylcholine glycol acrylate² | |
| water (and) phenyl trimethicone (and) | 1-10 |
| cylcomethicone (and) phospholipids (and) | |
| dimethiconol (and) polyphosphorylcholine | |
| glycol acrylate³ | |
| water (and) hydrogenated polyisobutene (and) | 1-10 |
| PEG-8 (and) cylcomethicone (and) | |
| phospholipids (and) polyphosphorylcholine | |
| glycol acrylate⁴ | |
| Preservative, fragrance and color | q.s. |
| Water | to 100.00 |

¹Acid Stable Base Powder;
²AM200;
³AM300;
⁴AM400;
(from Collaborative Laboratories, Inc. Stony Brook, NY)

### EXAMPLE 31

| INCI Name | w/w (%) |
|---|---|
| DFMO | 0.5-15 |
| Glycerol (Glycerin) | 0-5 |
| Isoceteth-20 | 3-7 |
| Glyceryl Isostearate | 1.5-5 |
| Dicaprylyl ether | 3-15 |
| Hydroxypropyl starch phosphate (and) | 1.0-5.0 |
| acrylates/vinyl isodecanoate crosspolymer | |
| (and) xanthan gum (and) ceratonia siliqua | |
| gum (and) cyamopsis tetragonoloba (guar) | |
| gum.¹ | |
| water (and) cyclomethicone (and) PEG-8 | 1-10 |
| (and) phospholipids (and) | |
| polyphosphorylcholine glycol acrylate² | |
| water (and) phenyl trimethicone (and) | 1-10 |
| cylcomethicone (and) phospholipids (and) | |
| dimethiconol (and) polyphosphorylcholine | |
| glycol acrylate³ | |
| water (and) dimethicone/vinyl dimethicone | 1-10 |
| crosspolymer (and) PEG-8 (and) | |
| cylcomethicone (and) phospholipids (and) | |
| polyphosphorylcholine glycol acrylate⁴ | |
| Preservative, fragrance and color | q.s. |
| Water | to 100.00 |

¹Acid Stable Base Powder;
²AM200;
³AM300;
⁴AM600;
(from Collaborative Laboratories, Inc. Stony Brook, NY)

Examples 1-5 and 18-22 generally can be replicated with other hydrophilic active compounds that are inhibitors of hair growth in place of DFMO.

For comparison purposes, a cream formulation (as shown in Table 1) was prepared as described in U.S. Pat. No. 5,648,394. Briefly, a water phase that included deionized water and DFMO, and an oil phase that included glyceryl stearate, PEG 100, cetearyl alcohol, ceteareth-20, mineral oil, stearyl alcohol and dimethicone, were heated to 70° C. At 70° C., the oil phase was added to the water phase and mixed for 20 minutes. The emulsion was cooled down to 40-45° C. and then preservatives were added.

Also for comparison purpose, a hydroalcoholic formulation was prepared as described in U.S. Pat. No. 5,132,293. For this formulation, the hydroalcoholic vehicle was prepared by mixing the components listed under hydroalcoholic formulation (HA) in Table 1 (below). DFMO was added to this solution to achieve a desired concentration, and the solution was mixed until complete dissolution occurred.

TABLE 1
Components of Two Standard Formulations without DFMO

| Hydroalcoholic Formulation (HA)^{a} | | Cream Formulation (CR)^{b} | |
|---|---|---|---|
| Water DI | 68% | Water DI | 80% |
| Ethanol | 16% | Glyceryl Stearate | 4% |
| Propylene Glycol | 5% | PEG-100 | 4% |
| Dipropylene Glycol | 5% | Cetearyl Alcohol | 3% |
| Benzyl Alcohol | 4% | Ceteareth-20 | 2.5% |
| Propylene Carbonate | 2% | Mineral Oil | 2% |
| | | Stearyl Alcohol | 2% |
| | | Dimethicone | 0.5% |
| | | Preservative | <1% |

^{a}U.S. Pat. No. 5,132,293;
^{b}U.S. Pat. No. 5,648,394.

The composition should be topically applied to a selected area of the body from which it is desired to reduce hair growth. For example, the composition can be applied to the face, particularly to the beard area of the face, i.e., the cheek, neck, upper lip, or chin. The composition also may be used as an adjunct to other methods of hair removal, for example, shaving, waxing, mechanical epilation, chemical depilation, and electrolysis.

The composition can also be applied to the legs, arms, torso or armpits. The composition is particularly suitable for reducing the growth of unwanted hair in women, particularly unwanted facial hair, for example, on the upper lip or chin. The composition should be applied once or twice a day, or even more frequently, to achieve a perceived reduction in hair growth. Perception of reduced hair growth can occur as early as 24 hours or 48 hours (for instance, between normal shaving intervals) following use or can take up to, for example, three months. Reduction in hair growth is demonstrated when, for example, the rate of hair growth is slowed, the need for removal is reduced, the subject perceives less hair on the treated site, or quantitatively, when the weight of hair removed (i.e., hair mass) is reduced (quantitatively), subjects perceive a reduction, for example, in facial hair, or subjects are less concerned or bothered about their unwanted hair (e.g., facial hair).

The emulsion prepared using the phase inversion procedure can be formulated in different ways based on the potential site of application. For example, the emulsion can be formulated as a hydroalcoholic splash, after-shave lotion or quick-breaking foam for hair growth control on the male face. In addition, the emulsions are also suitable as a lotion, breaking foam and as disposable wipes for a hair growth control product on female legs.

Some of the examples described above were tested in various assays. The assay procedures will be described first, followed by the results.

Skin Penetration Assay

Dorsal skin from Golden, Syrian hamsters was clipped with electric clippers, trimmed to the appropriate size, and placed in a glass diffusion chamber. The receptor fluid consisted of phosphate buffered saline, an isotonic solution for maintaining cell viability and 0.1% sodium azide (a preservative) and was placed in the lower chamber of the diffusion apparatus such that the level of the receptor fluid was in parallel with the mounted skin. After equilibration at 37° C. for at least 30 minutes, 25 μl of the test or control formulation containing equal amounts of DFMO were added to the surface of the skin and gently spread over the entire surface with a glass stirring rod. A radiotracer amount of ³H-DFMO (0.5-1 microCurie per diffusion chamber) was used in the formulations to assess DFMO penetration. Penetration of DFMO was determined by removing an aliquot (400 μL) periodically throughout the course of the experiment, and quantitating radioactivity using liquid scintillation.

Hair Mass Reduction Assay

The method employs the use of Golden Syrian hamsters. Animals were housed individually in stainless steel cages and fed a Purina Certified diet and water ad libitum. Ten week-old male hamsters were assigned to groups of 16 and hair on both sides of the back was removed with surgical clippers (No. 40 blade). Each animal was fitted with an Elizabethan collar to prevent possible ingestion of test formulations. Animals were housed in a room with a controlled environment with temperatures between 18° C. and 26° C. with a relative humidity of 30% to 70%. In addition, a 14/10-hour, light/dark cycle is maintained and 10 or more air changes per hour will occur.

Topical administration of test formulations occurred once per day, Monday-Friday, for a total of 13 doses per site. Ten microliters of the formulation were applied topically to each flank organ and gently spread with a pipet tip. Typically, formulations containing DFMO were applied to the left flank organ and the vehicle control formulation applied to the right flank organ. In situations where an accumulation of residue is noted, both flank organs on that animal (treated and vehicle control) were washed with warm water prior to the next dose. About 24 hours after the last treatment the animals were euthanized. Dorsal skin including the region around the flank organs was trimmed and final observations made. Flank organ hairs were harvested with a scalpel and weighed. Hair mass reduction was determined by calculating the mean % inhibition of hair mass for the group (typically comprised of 8 animals).

Hair Follicle Spatial Mass Assay

After completion of the hair mass assay, the remaining skins are placed surface side down and a small amount of glycerin is applied to the underside of the flank organs. After setting for 5 minutes to permit clarification of the tissue, the flank organ undersides were imaged under a dissecting microscope with a magnification of 10×. Lighting conditions were held constant within an animal, but, may be adjusted in between animals since there is variability between animals that in some cases requires more or less light. Images were then quantitated using IMAQ Vision Builder (National Instruments, Inc.) software. The software measures the intensity of light passing through the image within a selected area. The greater the intensity, the fewer number or smaller size of follicles are present, thus, more atrophy has occurred.

Ornithine Decarboxylase Assay

Hamster flank organs were homogenized in a buffer containing 50 mM sodium phosphate, pH 7.2, 0.4 mM pyridoxal phosphate, 4 mM dithiothreitol and 1 mM EDTA. The homogenates were then centrifuged at 12,000× g for 5 minutes at 4° C. to generate the soluble ODC supernatant. Typically, 2 flank organs were pooled together to give a protein concentration of 2 mg/ml. This supernatant was used as the source of enzyme in the ODC assay. A previously described (Kozumbo et al. Cancer Res. 43: 2255-2259, 1983) radiometric assay for ODC was used for determining enzymatic activity in hamster flank organ and human hair follicles. This assay measures the release of ¹⁴CO₂ from L-[1-¹⁴C]ornithine hydrochloride in the presence of the cofactor pyridoxal phosphate. The reaction mixture included a 10 μl aliquot of 50 mM sodium phosphate, pH 7.2; 1 mM EDTA; 0.2 mM pyridoxal phosphate; 4 mM dithiothreitol; 0.4 mM L-omithine, and up to 0.5 μCi L-[1-¹⁴C]ornithine hydrochloride. The reaction was initiated with the addition of 20 μl of supernatant from the tissue homogenate and, at the same time, 5 μl of 40% KOH was deposited in the underside of the lid of the Eppendorf tube. The reaction was carried out at 37° C. for up to three hours whereupon the reaction mixture was heated to 95° C. for 2 minutes and then set overnight at room temperature. Eppendorf lids were removed and placed in scintillation vials containing 12 mls of Econoscint and 100 μl acetic acid. The release of ¹⁴CO₂ was quantitated using liquid scintillation.

### Results

Skin Penetration Assay

Skin penetration of DFMO was enhanced when the composition included an emulsion prepared by a phase inversion process. Initially, two emulsions with different particle sizes, a droplet size (<100 nm) that was transparent (Example 1) and a larger droplet size (>100 nm) that was milky in appearance (Example 4) were studied. The example 1 formulation produced a 3-fold increase in skin penetration of DFMO, based on % of applied dose in the receptor compartment, when compared to the cream CR formulation, as shown in Table 2A. In addition, the rate of DFMO penetration was determined for each formulation and the results are shown in Table 2B, where it can be seen that the Example 1 formulation exhibited a 2.90-fold increase in DFMO penetration rate versus the cream CR formulation.

TABLE 2A
Increased DFMO (1%) Skin Penetration in
CR Cream and Example 1 Formulations

| | % Applied Dose | | |
|---|---|---|---|
| Time (hrs) | CR | Example 1 | Fold-Increase |
| 2 | 0.69 ± .11 | 1.05 ± .43 | 1.52 |
| 4 | 0.90 ± .12 | 1.78 ± .69 | 1.98 |
| 6 | 1.26 ± .14 | 2.55 ± .85 | 2.02 |
| 24 | 2.49 ± .22 | 7.06 ± 1.69 | 2.83 |

TABLE 2B
Increased Rate of ³H-DFMO Penetration through
Hamster Skin Using Example 1 Delivery System

| | Rate of Skin Penetration | | | |
|---|---|---|---|---|
| Time Range | CR | Example 1 | Fold Increase | p value |
| 0-6 hr | 0.21 ± .01 | 0.43 ± .10 | 2.05 ± .70 | 0.069 |
| 0-24 hr | 0.10 ± .01 | 0.29 ± .06 | 2.90 ± .78 | 0.024 |

Rate is expressed as % applied dose/hour x cm²; ±values represent sem; p values were determined using a paired t test. DFMO concentration was 1% in both

In a similar experiment, the DFMO penetration enhancement properties of the Example 1 formulation were compared with the HA formulation. As depicted in Table 3A, about a 4-fold increase in the penetration of DFMO was demonstrated with the Example 1 formulation over the HA formulation 24 hours following skin application. Analysis of the rate of penetration also revealed a nearly 4-fold increase in the rate of DFMO penetration from the Example 1 formulation (Table 3B) over a 24-hour period versus the HA formulation.

TABLE 3A
DFMO (1%) Skin Penetration in HA and Example 1 Formulations

| | % Applied Dose | | |
|---|---|---|---|
| Time (hrs) | HA | Example 1 | Fold-Increase |
| 2 | 0.63 ± .40 | 0.74 ± .71 | 1.17 |
| 6 | 1.3 ± .57 | 2.37 ± .99 | 1.82 |
| 24 | 2.15 ± .85 | 8.32 ± 1.92 | 3.87 |

TABLE 3B
Increased Rate of ³H-DFMO Penetration through
Hamster Skin Using Example 1 Delivery System

| | Rate of Skin Penetration | | | |
|---|---|---|---|---|
| Time Range | HA | Example 1 | Fold Increase | p value |
| 0-6 hr | 0.22 ± .06 | 0.40 ± .11 | 1.82 ± .5 | 0.01 |
| 0-24 hr | 0.09 ± .03 | 0.35 ± .08 | 3.89 ± .4 | 0.0004 |

Rate is expressed as % of applied dose/hour×cm²: ±values represent sem; p values were determined using a paired t test. DFMO concentration was 1% in both formulations.

Example 4 was also evaluated for its ability to increase DFMO skin penetration versus the CR formulation. Example 4 differs from the Example 1 formulation with respect to the dicaprylyl ether (Cetiol OE, Cognis) concentration, namely with Example 4 containing 15% versus 5% for the Example 1 formulation—the balance of which is made up with water. FIG. 4A shows significant enhancement of DFMO penetration through the skin and Table 4B highlights the corresponding increase in the rate of DFMO penetration. In each case, the Example 4 formulation exhibited about a 3.3-fold increase versus the CR formulation.

TABLE 4A
DFMO (1%) Skin Penetration in CR and Example 4 Formulations

| | % Applied Dose | | |
|---|---|---|---|
| Time (hrs) | CR | Example 4 | Fold-Increase |
| 2 | 0.85 ± .15 | 0.98 ± .16 | 1.15 |
| 6 | 1.14 ± .15 | 2.44 ± .16 | 2.14 |
| 24 | 3.03 ± .52 | 10.2 ± .89 | 3.37 |

TABLE 4B
Increased Rate of ³H-DFMO Penetration through
Hamster Skin Using Example 4 Delivery System

| | Rate of Skin Penetration | | | |
|---|---|---|---|---|
| Time Range | CR | Example 4 | Fold Increase | p value |
| 0-6 hr | 0.19 ± .02 | 0.41 ± .07 | 2.16 | 0.003 |
| 0-24 hr | 0.13 ± .02 | 0.43 ± .04 | 3.31 | 0.003 |

Rate is expressed as % applied dose/hour×cm²; ±values represent sem; p values were determined using a paired t test. DFMO concentration was 1% in both formulations.

These data suggest that skin penetration can be significantly increased with the compositions including an emulsion prepared using a phase inversion procedure. Furthermore, the range of droplet size tested produced significant enhancement in skin penetration.

Several additional examples also were assayed for their ability to increase the skin penetration of DFMO. These formulations are similar in that they all have between 75 and 85% water, 3% glycerol and relatively low levels of surfactants and co-surfactants. As shown in Table 5 at least a 2-fold increase in DFMO skin penetration was obtained with each of the examples as compared to the HA formulation with Example 1, Example 2, and Example 4 demonstrating the most pronounced increases in DFMO penetration.

TABLE 5
Increased Skin Penetration of DFMO (1%) with
Six Examples Compared with HA Formulation.

| | % Applied Dose | | |
|---|---|---|---|
| Formulation | HA | Formulation | Fold-Increase |
| Example 1 | 2.15 ± .85 | 8.32 ± 1.92 | 3.9 |
| Example 2 | 2.12 ± .62 | 7.71 ± 1.54 | 3.6 |
| Example 3 | 2.42 ± .88 | 5.56 ± 1.54 | 2.3 |
| Example 4 | 3.03 ± .52 | 10.26 ± .89 | 3.4 |
| Example 5 | 1.94 ± .78 | 4.3 ± 1.14 | 2.2 |
| Example 6 | 2.64 ± 1.63 | 5.22 ± 1.35 | 2.0 |

Hair Growth Assay

An increase in hair mass reduction efficacy with 1% DFMO was demonstrated in two separate assays for Example 1, in comparison to the HA formulation. Table 6 depicts a 34% hair mass reduction with the HA formulation and a 67% reduction with the Example 1 formulation, representing a 2-fold increase in DFMO mediated hair mass reduction efficacy between the formulations. Similarly, Table 7 shows a 2-fold increase in efficacy with Example 1 over the CR formulation and, interestingly, shows that 1% DFMO in Example 1 gives rise to a similar degree of efficacy achieved by 10% DFMO in the HA formulation. In total, Example 1 containing 1% DFMO was evaluated in 3 separate hair mass assays and demonstrated similar findings in each test.

TABLE 6
Hair Mass Reduction by 1% DFMO in HA and Example 1
Formulations

% Hair Mass Reduction

| HA | Example 1 | Fold-Increase |
|---|---|---|
| 34 ± 12 | 67 ± 10 | 1.97 |

Values represent the mean of two experiments each conducted with 8 animals per group.

TABLE 7
Hair Mass Reduction by 1% DFMO in CR and Example 1
Formulations and by 10% DFMO in HA Formulation.

| Formulation* | DFMO Concentration | % Hair Mass Reduction |
|---|---|---|
| CR | 1% | 28 ± 11 |
| Example 1 | 1% | 57 ± 9 |
| HA | 10% | 54 ± 8 |

*All three of these formulations were evaluated concurrently in the same hair mass reduction assay.

In addition to being evaluated in a hair mass assay where each animal receives a DFMO-containing formulation on one flank organ and a vehicle control formulation on the contra-lateral flank organ, Example 1 was also compared with the HA formulation in an experiment designed determine differences within a single animal. For example, the Example 1 formulation was applied to one flank organ (left) and the HA formulation was applied to contra-lateral flank organ (right). As shown in Table 8, the Example 1 formulation produced a 40% greater inhibition of hair mass than the HA formulation.

TABLE 8
Hair Mass Reduction by 1% DFMO in HA and Example 1

| Hair Mass (mg) | | |
|---|---|---|
| Example 1 | | |
| (Left Flank Organ) | HA (Right Flank Organ) | % Hair Mass Reduction* |
| 0.99 ± .25 | 1.68 ± .22 | 40 ± 13 |

*The determination of hair mass reduction is based on differences within the same animal (n = 8).

Several other examples were also shown to increase the level of hair mass reduction when compared with the HA formulation, as depicted in Table 9. These examples differ primarily with respect to their emollient oils, which included diacaprylyl ether, cyclomethicone, caprylic/capric triglyceride, diethylcyclohexane and/or coco-caprylate/caprate.

TABLE 9
Hair Mass Reduction by 1% DFMO in HA and Examples

| | | % Increase |
|---|---|---|
| | | Compared with |
| Formulation | % Hair Mass Reduction | Untreated Control |
| HA | 22 ± 11 | — |
| Example 1 | 40 ± 13 | 181 |
| Example 2 | 34 ± 2 | 155 |
| Example 7 | 54 ± 5 | 245 |
| Example 3 | 30 ± 9 | 136 |
| Example 5 | 49 ± 9 | 223 |

Hair Follicle Spatial Mass Assay

Another effect of DFMO on flank organ, hair follicles is the induction of shrinkage or atrophy. Studies were conducted to quantify the difference in atrophy induction between the HA and Example 1 formulations, following topical application. A significant difference between the HA formulation and Example 1 and Example 5 was demonstrated. Tables 10 and 11 show a significant induction in average light intensity with Example 1 when compared with the HA formulation. Intensity represents the amount of light that passes through the flank organ image, thus a darker image will have a lower mean intensity—indicative of more hair follicles and/or larger hair follicles. The lighting conditions and magnification as well as the area of the image measured were held constant within an animal. However, due to animal to animal variation in the size and pigment of the flank organ underside, inter-animal comparisons can only be made on the change in mean intensity and not on the absolute intensity values.

TABLE 10
Induction of Hair Follicle Atrophy with 1% DFMO in Example 1

Hair Follicle
Atrophy (intensity)

| Formulation | DFMO | Vehicle | % Induction | P value |
|---|---|---|---|---|
| HA | 235 ± 4 | 235 ± 8 | 0 ± 3 | — |
| Example 1 | 185 ± 3 | 146 ± 10 | 21 ± 5 | 0.03 |

P values were determined using a t test to compare the intensity between DFMO-treated and vehicle-treated flank organs for each formulation.

TABLE 11
Flank Organ Hair Follicle Atrophy following Topical
Application of 1% DEMO in HA, Example 1 and Example
5 Formulations.

| | % Hair Follicle Atrophy | |
|---|---|---|
| Formulation | Induction | Fold-Increase vs HA |
| HA | 5 ± 3 | — |
| Example 1 | 12 ± 3 | 2.4 |
| Example 5 | 13 ± 2 | 2.6 |

Ornithine Decarboxylase Assay

DFMO inhibits omithine decarboxylase, the enzyme that catalyzes the rate-limiting step in de novo synthesis of polyamines. The hamster flank organs were treated topically for 3 weeks with 1% DFMO in either the HA formulation or Example 1, whereupon flank organs were removed and assayed for ODC activity. ODC activity was inhibited by 22±10% with the HA formulation and inhibited by 66±3% with Example 1. The difference in the magnitude of inhibition was significantly increased with Example 1 as shown in Table 12. The level of inhibition obtained with 1% DFMO in Example 1 is similar to that obtained with 15% DFMO in the CR formulation.

TABLE 12
Inhibition of Flank Organ ODC Catalytic Activity after Topical
Treatment of 1% DFMO in HA and Example 1 Formulations

| | ODC Activity | | | |
|---|---|---|---|---|
| | (pmoles/hr × mg) | | | |
| Formulation | DFMO | Vehicle | % Inhibition | p Value |
| HA | 188 ± 14 | 264 ± 23 | 22 ± 10 | — |
| Example 1 | 52 ± 6 | 159 ± 14 | 66 ± 3 | 0.002 |

P values were determined using a paired t test.

Other embodiments are within the scope of the following claims.

What is claimed is:

1. A method of reducing human hair growth, comprising
selecting an area of skin from which reduced hair growth is desired, and applying to the area of skin, in an amount effective to reduce hair growth, a composition comprising an emulsion, including a compound that inhibits hair growth, wherein the emulsion has been prepared using a phase inversion procedure.

2. A method of reducing human hair growth, comprising
selecting an area of skin from which reduced hair growth is desired, and applying to the area of skin, in an amount effective to reduce hair growth, a composition comprising an emulsion including a compound that inhibits hair growth, the emulsion including droplets having an average size of from 10 nm to 150 nm.

3. The method of claims 1 or 2, wherein the emulsion is an oil-in-water emulsion in which oil droplets are dispersed in a water phase.

4. The method of claim 3, wherein the oil droplets have an average size of from 25 nm to 100 nm.

5. The method of claim 3, wherein the droplets are sufficiently small that the emulsion is clear.

6. The method of claims 1 or 2, wherein the compound is α-difluoromethylornithine.

7. The method of claim 6, wherein the α-difluoromethylornithine comprises at least 80% L-α-difluoromethylornithine.

8. The method of claim 6, wherein the composition comprises from 1% to 20% by weight α-difluoromethylornithine.

9. The method of claims 1 or 2, wherein the compound is selected from the group consisting of HMG CoA reductase inhibitors, NO synthetase inhibitors, lipoxygenase inhibitors, cyclooxygenase inhibitors, sulffiydryl active compounds, anti-angiogenic agents, matrix metalloproteinase inhibitors, protein kinase C inhibitors, and catechin derivatives.

10. The method of claims 1 or 2, wherein the compound is selected from the group consisting of simvastatin; atorvastatin; lovastatin; fluvastatin; mevastatin; N^{G}-methyl-L-arginine; N^{G}-nitro-L-arginine; benzoyl-L-argininamide; L-argininamide; quercetin; apigenin; nordihydroguaratic acid; ketoprofen; naproxen; tolmetin; diclofenac; diflunisal; sulindac; thiosalicylic acid; cysteamine; diethyldithiocarbamic acid; D-peanicillamine; N-acetyl-L-cysteine; bathocuproine; enalapril; tamoxifen; cimetidine; mycophenolic acid; tetracycline; doxycycline; minocycline; verapamil; thioridazine; trifluoperizine; 1-(5-isoquinolinylsulfonyl)-2-methylpiperazine; glycyrrhetinic acid; epigallocatechin gallate; epicatechin gallate; epigallocatechin; epicatechin; fusidic acid; and nitroso-acetyl-penicillamine.

11. The method of claim 3, wherein the composition comprises from 1% to 30% of the oil phase and comprises from 40% to 99% of the water phase by weight.

12. The method of claims 1 or 2, wherein the composition is a liquid.

13. The method of claims 1 or 2, wherein the composition is a cream.

14. The method of claims 1 or 2, wherein the composition comprises an emollient.

15. The method of claim 14, wherein the emollient is selected from the group consisting of stearyl alcohol, mink oil, cetyl alcohol, oleyl alcohol, isopropyl laurate, polyethylene glycol, petroleum jelly, palmitic acid, oleic acid, and myristyl myristate.

16. The method of claim 3, wherein the water phase comprises, in addition to water, a component selected from the group consisting of ethyl alcohol, isopropanol, acetone, diethylene glycol, ethylene glycol, dimethyl sulfoxide, and dimethyl formamide.

17. The method of claim 3, further comprising an emulsifier having the formula R-O—[(CH₂)ₓ—O]ₙ—H, wherein x is 2 or 3, n is from 5 to 50, and R is an alkyl or alkylene group having from 5 to 30 carbon atoms.

18. The method of claim 17, wherein the emulsifier is selected from the group consisting of polyethylene glycol (13-20) stearyl ether, polyethylene glycol (12-20) isostearyl ether, polyethylene glycol (13-20) cetyl ether, polyethylene glycol (13-20) isocetyl ether, polyethylene glycol (12-15) oleyl ether, polyethylene glycol (12) lauryl ether, polyethylene glycol (13-20) cetylstearyl ether, polyethylene glycol (20-25) stearate, polyethylene glycol (12-25) isostearate, polyethylene glycol (12-20) oleate, and polyethylene glycol (20-23) glyceryl laurate.

19. The method of claim 3, wherein the oil phase composition comprises an ester selected from the group consisting of (1) esters of an alkanecarboxylic acid having from 3 to 30 carbon atoms and alcohols having from 3 to 30 carbon atoms, and (2) esters of aromatic carboxylic acids and alcohols having from 3 to 30 carbon atoms.

20. The method of claim 19, wherein the ester is selected from the group consisting of glyceryl isostearate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-hexyldecyl stearate, 2-octyldodecyl palmitate, oleyl oleate, oleyl erucate, erucyl oleate, and erucyl erucate.

21. The method of claim 3, wherein the oil phase comprises a compound selected from the group consisting of 2-ethylhexyl isostearate, octyldodecanol, isotridecyl isononanoate, isoeicosane, 2-ethylexyl cocoate, C12-15 alkyl benzoate, caprylic-capric acid triglyceride, and dicaprylyl ether.

22. The method of claim 1, wherein the area of skin is on the face and/or neck of the human.

23. The method of claims 1 or 2, wherein the composition includes a penetration enhancer selected from the group consisting of a polyoxyethylene ether having the chemical formula R(OCH₂CH₂)_{b}OH, where R is a saturated or unsaturated alkyl group including from 6 to 22 carbon atoms and b is from 2 to 200; mineral oil; cis-fatty acids; fatty acid esters; terpenes; non-ionic surfactants; 2-n-nonyl-1,3-dioxolane; film-forming agents; dipropylene glycol dimethylethes; cetiol; capric/caprylic triglyceride; triacetin monocaprylate/caprate; and 1-dodecyl-2-pyrrolidone.

24. A method of reducing human hair growth, comprising
selecting an area of skin from which reduced hair growth is desired; and
applying to the area of skin, in an amount effective to reduce hair growth, a composition comprising an emulsion including a compound that inhibits hair growth, the emulsion including droplets sufficiently small that the composition is substantially clear.

25. The method of claim 24, wherein the emulsion is an oil-in-water emulsion.

26. A composition for reducing hair growth, the composition comprising an oil-in-water emulsion comprising a compound that inhibits hair growth, the emulsion including an oil phase dispersed as droplets having an average size of from 10 nm to 150 nm in a water phase.

27. The composition of claim 26, wherein the compound is α-difluoromethylornithine.

28. The composition of claim 26, wherein the oil phase droplets have an average size of from 25 nm to 100 nm.

29. The composition of claim 27, wherein the composition includes from 1% to 20% by weight of α-difluoromethylornithine.

30. The composition of claims 26 or 29, wherein the oil composition comprises from 1% to 30% by weight of the oil phase and from 40% to 99% of the water phase.

31. The composition of claim 30, wherein the oil phase comprises a component selected from the group consisting of isoceteth-20, glyceryl isostearate, and dicaprylyl ether.

32. The composition of claim 27, wherein the composition includes from 2% to 10% isoceteth-20 by weight; from 0.5% to 10% glyceryl isostearate by weight; and from 5% to 30% dicaprylyl ether by weight.

33. The composition of claim 32, wherein the composition further comprises from 1% to 10% glycerol by weight.

34. The composition of claim 26, wherein the droplets are sufficiently small that the composition is substantially clear.

35. The composition of claim 26, wherein the oil phase comprises glyceryl isostearate.

36. The composition of claim 35, wherein the composition further comprises an emulsifier having the formula R-O—[(CH₂)ₓ—O]ₙ—H, wherein x is 2 or 3, n is from 5 to 50, and R is an alkyl or alkylene group having from 5 to 30 carbon atoms.

37. The composition of claim 36, wherein the emulsifier is isoceteth 20.

38. The composition of claim 26, wherein the oil phase further includes an emollient.

39. The composition of claim 38, wherein the emollient is selected from the group consisting of dicaprylyl ether, caprylic/capric triglyceride, coco-caprylate, oleyl alcohol, and bis(2-ethylhexyl)carbonate.

40. The composition of claims 35-39, wherein the component is α-difluoromethylornithine.

41. A method of making an oil-in-water emulsion comprising a compound that inhibits hair growth, said emulsion including an oil phase and a water phase that includes said compound dissolved therein, said method comprising mixing the oil phase and the water phase together at a temperature at or above a phase inversion temperature for the emulsion to form a water-in-oil emulsion, then cooling the water-in-oil emulsion to a temperature below the phase inversion temperature to cause the water-in-oil emulsion to invert to an oil-in-water emulsion.

42. The method of claim 41, wherein the compound is α-difluoromethylornithine.

43. A method of reducing human hair growth comprising
selecting an area of skin from which reduced hair growth is desired; and
applying to the area of skin, in an amount effective to reduce hair growth, a composition comprising a nanoemulsion including a compound that inhibits hair growth.

44. The method of claim 43, wherein the compound is α-difluoromethylornithine.

45. A topical hair growth inhibiting composition comprising an oil-in-water emulsion, said emulsion comprising an oil phase including glyceryl isostearate and a water phase including a compound that inhibits hair growth dissolved therein.

46. The composition of claim 45, wherein the composition further comprises an emulsifier having the formula R-O—[(CH₂)ₓ—O]ₙ—H, wherein x is 2 or 3, n is from 5 to 50, and R is an alkyl or alkylene group having from 5 to 30 carbon atoms.

47. The composition of claim 36, wherein the emulsifier is isoceteth 20.

48. The composition of claim 45, wherein the oil phase further comprises an emollient.

49. The composition of claim 48, wherein the emollient is selected from the group consisting of dicaprylyl ether, caprylic/capric triglyceride, coco-caprylate, oleyl alcohol, and bis(2-ethylhexyl)carbonate.

50. The composition of claims 46-49, wherein the compound is α-difluoromethylornithine.
